SEQUENCE LISTING (116 sequences)

SEQ ID NO: 1 - protein, 18 aa, synthetic construct
HKNNKSWMESEFRVYSSA

SEQ ID NO: 2 - protein, 16 aa, synthetic construct
KHTPINLVRDLPQGFS

SEQ ID NO: 3 - protein, 13 aa, synthetic construct
HRSYLTPGDSSSG

SEQ ID NO: 4 - protein, 12 aa, synthetic construct
YGVSPTKLNDLC

SEQ ID NO: 5 - protein, 23 aa, synthetic construct
RGDEVRQIAPGQTGKIADYNYKL

SEQ ID NO: 6 - protein, 22 aa, synthetic construct
APGQTGKIADYNYKLPDDFTGC

SEQ ID NO: 7 - protein, 29 aa, synthetic construct
SKVGGNYNYLYRLFRKSNLKPFERDISTE

SEQ ID NO: 8 - protein, 23 aa, synthetic construct
KSNLKPFERDISTEIYQAGSTPC

SEQ ID NO: 9 - protein, 22 aa, synthetic construct
CYFPLQSYGFQPTNGVGYQPYR

SEQ ID NO: 10 - protein, 13 aa, synthetic construct
CGPKKSTNLVKNK

SEQ ID NO: 11 - protein, 12 aa, synthetic construct
TTDAVRDPQTLE

SEQ ID NO: 12 - protein, 23 aa, synthetic construct
IKDFGGFNFSQILPDPSKPSKRS

SEQ ID NO: 13 - protein, 20 aa, synthetic construct
YDPLQPELDSFKEELDKYFK

SEQ ID NO: 14 - protein, 14 aa, synthetic construct
KNHTSPDVDLGDIS

SEQ ID NO: 15 - protein, 13 aa, synthetic construct
CKFDEDDSEPVLK

SEQ ID NO: 16 - protein, 12 aa, synthetic construct
GVYYHKNNKSWM

SEQ ID NO: 17 - protein, 18 aa, synthetic construct
GVYYHKNNKSWMESEFRV

SEQ ID NO: 18 - protein, 27 aa, synthetic construct
GVYYHKNNKSWMESEFRVYSSANNCTF

SEQ ID NO: 19 - protein, 18 aa, synthetic construct
VYYHKNNKSWMESEFRVY

SEQ ID NO: 20 - protein, 15 aa, synthetic construct
YHKNNKSWMESEFRV

SEQ ID NO: 21 - protein, 23 aa, synthetic construct
HKNNKSWMESEFRVYSSANNCTF

SEQ ID NO: 22 - protein, 30 aa, synthetic construct
KSWMESEFRVYSSANNCTFEYVSQPFLMDL

SEQ ID NO: 23 - protein, 20 aa, synthetic construct
KIYSKHTPINLVRDLPQGFS

SEQ ID NO: 24 - protein, 15 aa, synthetic construct
KHTPINLVRDLPQGF

SEQ ID NO: 25 - protein, 18 aa, synthetic construct
FSALEPLVDLPIGINITR

SEQ ID NO: 26 - protein, 26 aa, synthetic construct
FSALEPLVDLPIGINITRFQTLLALH

SEQ ID NO: 27 - protein, 18 aa, synthetic construct
KLNDLCFTNVYADSFVIR

SEQ ID NO: 28 - protein, 18 aa, synthetic construct
RGDEVRQIAPGQTGKIAD

SEQ ID NO: 29 - protein, 20 aa, synthetic construct
RGDEVRQIAPGQTGKIADYN

SEQ ID NO: 30 - protein, 26 aa, synthetic construct
RGDEVRQIAPGQTGKIADYNYKLPDD

SEQ ID NO: 31 - protein, 14 aa, synthetic construct
VRQIAPGQTGKIAD

SEQ ID NO: 32 - protein, 27 aa, synthetic construct
VRQIAPGQTGKIADYNYKLPDDFTGCV

SEQ ID NO: 33 - protein, 18 aa, synthetic construct
RQIAPGQTGKIADYNYKL

SEQ ID NO: 34 - protein, 26 aa, synthetic construct
RQIAPGQTGKIADYNYKLPDDFTGCV

SEQ ID NO: 35 - protein, 23 aa, synthetic construct
APGQTGKIADYNYKLPDDFTGCV

SEQ ID NO: 36 - protein, 23 aa, synthetic construct
GNYNYLYRLFRKSNLKPFERDIS

SEQ ID NO: 37 - protein, 26 aa, synthetic construct
GNYNYLYRLFRKSNLKPFERDISTEI

SEQ ID NO: 38 - protein, 19 aa, synthetic construct
NYNYLYRLFRKSNLKPFER

SEQ ID NO: 39 - protein, 20 aa, synthetic construct
YRLFRKSNLKPFERDISTEI

SEQ ID NO: 40 - protein, 15 aa, synthetic construct
KSNLKPFERDISTEI

SEQ ID NO: 41 - protein, 26 aa, synthetic construct
KSNLKPFERDISTEIYQAGSTPCNGV

SEQ ID NO: 42 - protein, 27 aa, synthetic construct
VEGFNCYFPLQSYGFQPTNGVGYQPYR

SEQ ID NO: 43 - protein, 17 aa, synthetic construct
NCYFPLQSYGFQPTNGV

SEQ ID NO: 44 - protein, 28 aa, synthetic construct
CYFPLQSYGFQPTNGVGYQPYRVVVLSF

SEQ ID NO: 45 - protein, 18 aa, synthetic construct
RDIADTTDAVRDPQTLEI

SEQ ID NO: 46 - protein, 26 aa, synthetic construct
VEQDKNTQEVFAQVKQIYKTPPIKDF

SEQ ID NO: 47 - protein, 27 aa, synthetic construct
IYKTPPIKDFGGFNFSQILPDPSKPSK

SEQ ID NO: 48 - protein, 29 aa, synthetic construct
IYKTPPIKDFGGFNFSQILPDPSKPSKRS

SEQ ID NO: 49 - protein, 14 aa, synthetic construct
TVYDPLQPELDSFK

SEQ ID NO: 50 - protein, 22 aa, synthetic construct
TVYDPLQPELDSFKEELDKYFK

SEQ ID NO: 51 - protein, 24 aa, synthetic construct
TVYDPLQPELDSFKEELDKYFKNH

SEQ ID NO: 52 - protein, 35 aa, synthetic construct
TVYDPLQPELDSFKEELDKYFKNHTSPDVDLGDIS

SEQ ID NO: 53 - protein, 12 aa, synthetic construct
VYDPLQPELDSF

SEQ ID NO: 54 - protein, 21 aa, synthetic construct
VYDPLQPELDSFKEELDKYFK

SEQ ID NO: 55 - protein, 13 aa, synthetic construct
YDPLQPELDSFKE

SEQ ID NO: 56 - protein, 13 aa, synthetic construct
YDPLQPELDSFKE

SEQ ID NO: 57 - protein, 21 aa, synthetic construct
YDPLQPELDSFKEELDKYFKN

SEQ ID NO: 58 - protein, 22 aa, synthetic construct
YDPLQPELDSFKEELDKYFKNH

SEQ ID NO: 59 - protein, 33 aa, synthetic construct
YDPLQPELDSFKEELDKYFKNHTSPDVDLGDIS

SEQ ID NO: 60 - protein, 19 aa, synthetic construct
DPLQPELDSFKEELDKYFK

SEQ ID NO: 61 - protein, 20 aa, synthetic construct
DPLQPELDSFKEELDKYFKN

SEQ ID NO: 62 - protein, 18 aa, synthetic construct
PLQPELDSFKEELDKYFK

SEQ ID NO: 63 - protein, 19 aa, synthetic construct
PLQPELDSFKEELDKYFKN

SEQ ID NO: 64 - protein, 31 aa, synthetic construct
PLQPELDSFKEELDKYFKNHTSPDVDLGDIS

SEQ ID NO: 65 - protein, 21 aa, synthetic construct
EELDKYFKNHTSPDVDLGDIS

SEQ ID NO: 66 - protein, 25 aa, synthetic construct
EELDKYFKNHTSPDVDLGDISGINA

SEQ ID NO: 67 - protein, 18 aa, synthetic construct
KNHTSPDVDLGDISGINA

SEQ ID NO: 68 - protein, 10 aa, synthetic construct
FDEDDSEPVL

SEQ ID NO: 69 - protein, 18 aa, synthetic construct
HKNNKSWMESGFRVYSSA

SEQ ID NO: 70 - protein, 16 aa, synthetic construct
KHTPINLVRGLPQGFS

SEQ ID NO: 71 - protein, 23 aa, synthetic construct
RGDEVRQIAPGQTGNIADYNYKL

SEQ ID NO: 72 - protein, 23 aa, synthetic construct
RGDEVRQIAPGQTGTIADYNYKL

SEQ ID NO: 73 - protein, 22 aa, synthetic construct
APGQTGNIADYNYKLPDDFTGC

SEQ ID NO: 74 - protein, 22 aa, synthetic construct
APGQTGTIADYNYKLPDDFTGC

SEQ ID NO: 75 - protein, 29 aa, synthetic construct
SKVGGNYNYRYRLFRKSNLKPFERDISTE

SEQ ID NO: 76 - protein, 23 aa, synthetic construct
KSNLKPFERDISTEIYQAGSKPC

SEQ ID NO: 77 - protein, 22 aa, synthetic construct
CYFPLQSYGFQPTYGVGYQPYR

SEQ ID NO: 78 - protein, 18 aa, synthetic construct
GVYYHKNNKSWMESGFRV

SEQ ID NO: 79 - protein, 27 aa, synthetic construct
GVYYHKNNKSWMESGFRVYSSANNCTF

SEQ ID NO: 80 - protein, 18 aa, synthetic construct
VYYHKNNKSWMESGFRVY

SEQ ID NO: 81 - protein, 15 aa, synthetic construct
YHKNNKSWMESGFRV

SEQ ID NO: 82 - protein, 23 aa, synthetic construct
HKNNKSWMESGFRVYSSANNCTF

SEQ ID NO: 83 - protein, 30 aa, synthetic construct
KSWMESGFRVYSSANNCTFEYVSQPFLMDL

SEQ ID NO: 84 - protein, 20 aa, synthetic construct
KIYSKHTPINLVRGLPQGFS

SEQ ID NO: 85 - protein, 15 aa, synthetic construct
KHTPINLVRGLPQGF

SEQ ID NO: 86 - protein, 18 aa, synthetic construct
RGDEVRQIAPGQTGNIAD

SEQ ID NO: 87 - protein, 18 aa, synthetic construct
RGDEVRQIAPGQTGTIAD

SEQ ID NO: 88 - protein, 20 aa, synthetic construct
RGDEVRQIAPGQTGNIADYN

SEQ ID NO: 89 - protein, 20 aa, synthetic construct
RGDEVRQIAPGQTGTIADYN

SEQ ID NO: 90 - protein, 26 aa, synthetic construct
RGDEVRQIAPGQTGNIADYNYKLPDD

SEQ ID NO: 91 - protein, 26 aa, synthetic construct
RGDEVRQIAPGQTGTIADYNYKLPDD

SEQ ID NO: 92 - protein, 14 aa, synthetic construct
VRQIAPGQTGNIAD

SEQ ID NO: 93 - protein, 14 aa, synthetic construct
VRQIAPGQTGTIAD

SEQ ID NO: 94 - protein, 27 aa, synthetic construct
VRQIAPGQTGNIADYNYKLPDDFTGCV

SEQ ID NO: 95 - protein, 27 aa, synthetic construct
VRQIAPGQTGTIADYNYKLPDDFTGCV

SEQ ID NO: 96 - protein, 18 aa, synthetic construct
RQIAPGQTGNIADYNYKL

SEQ ID NO: 97 - protein, 18 aa, synthetic construct
RQIAPGQTGTIADYNYKL

SEQ ID NO: 98 - protein, 26 aa, synthetic construct
RQIAPGQTGNIADYNYKLPDDFTGCV

SEQ ID NO: 99 - protein, 26 aa, synthetic construct
RQIAPGQTGTIADYNYKLPDDFTGCV

SEQ ID NO: 100 - protein, 23 aa, synthetic construct
APGQTGNIADYNYKLPDDFTGCV

SEQ ID NO: 101 - protein, 23 aa, synthetic construct
APGQTGTIADYNYKLPDDFTGCV

SEQ ID NO: 102 - protein, 23 aa, synthetic construct
GNYNYRYRLFRKSNLKPFERDIS

SEQ ID NO: 103 - protein, 26 aa, synthetic construct
GNYNYRYRLFRKSNLKPFERDISTEI

SEQ ID NO: 104 - protein, 19 aa, synthetic construct
NYNYRYRLFRKSNLKPFER

SEQ ID NO: 105 - protein, 26 aa, synthetic construct
KSNLKPFERDISTEIYQAGSKPCNGV

SEQ ID NO: 106 - protein, 12 aa, synthetic construct
DVYYHKNNKSWM

SEQ ID NO: 107 - protein, 27 aa, synthetic construct
VKGFNCYFPLQSYGFQPTYGVGYQPYR

SEQ ID NO: 108 - protein, 17 aa, synthetic construct
NCYFPLQSYGFQPTYGV

SEQ ID NO: 109 - protein, 28 aa, synthetic construct
CYFPLQSYGFQPTYGVGYQPYRVVVLSF

SEQ ID NO: 110 - protein, 18 aa, synthetic construct
RDIDDTTDAVRDPQTLEI

SEQ ID NO: 111 - protein, 22 aa, synthetic construct
NYLYRLFRKSNLKPFERDISTE

SEQ ID NO: 112 - protein, 24 aa, synthetic construct
NYNYLYRLFRKSNLKPFERDISTE

SEQ ID NO: 113 - protein, 26 aa, synthetic construct
GGNYNYLYRLFRKSNLKPFERDISTE

SEQ ID NO: 114 - protein, 22 aa, synthetic construct
NYRYRLFRKSNLKPFERDISTE

SEQ ID NO: 115 - protein, 24 aa, synthetic construct
NYNYRYRLFRKSNLKPFERDISTE

SEQ ID NO: 116 - protein, 26 aa, synthetic construct
GGNYNYRYRLFRKSNLKPFERDISTE

(19) United States
(12) Patent Application Publication
(10) Pub. No.: US 2025/0099573 A1
(43) Pub. Date: Mar. 27, 2025

(54) SARS-COV-2 S PROTEIN POLYPEPTIDE ANTIGEN AND APPLICATION THEREOF

(71) Applicant: Chinese PLA General Hospital, Beijing (CN)

(72) Inventors: Fenghua XU, Beijing (CN); Shusen YAO, Beijing (CN); Qi SUN, Beijing (CN); Haoyuan SHI, Beijing (CN); Ying ZHANG, Beijing (CN); Zhuanqing HUANG, Beijing (CN); Yalong YANG, Beijing (CN)

(21) Appl. No.: 18/708,771

(22) PCT Filed: Nov. 3, 2022
(86) PCT No.: PCT/CN2022/129538

§ 371 Date: May 9, 2024

(30) Foreign Application Priority Data
Nov. 9, 2021 (CN) 202111320793.1

(51) Int. Cl. A61K 39/215; A61P 37/04; C07K 14/005; C07K 14/435

(52) U.S. Cl. CPC A61K 39/215; A61P 37/04; C07K 14/005; C07K 14/43509; C12N 2770/20022; C12N 2770/20034; C12N 2770/20071

(57) ABSTRACT

Provided are a polypeptide antigen derived from the S protein of SARS-CoV-2, a polypeptide vaccine containing the same, and applications thereof. The amino acid sequence of the polypeptide antigen provided by the present disclosure is as shown in any one of SEQ ID NOs: 1-116.

### CROSS-REFERENCE TO RELATED APPLICATIONS

This application is based on and claims priority to Chinese Patent Application No. 202111320793.1, filed on Nov. 9, 2021, the entire content of which is incorporated herein by reference.

### TECHNICAL FIELD

The present disclosure relates to technical field of molecular immunology, and specifically to a polypeptide antigen from the spike protein of Severe Acute Respiratory Syndrome Coronavirus 2 (SARS-CoV-2) and use thereof.

### BACKGROUND

Corona Virus Disease 2019 (CoVID-19), caused by infection of Severe Acute Respiratory Syndrome Coronavirus 2 (SARS-CoV-2), is currently raging around the world. As of Nov. 8, 2021, 250 million cases have infected SARS-CoV-2, and 5,059,356 lives were cumulatively claimed globally. In view of the rapid development of this pandemic and the fact that no specific drug has been found yet, a vaccine specific to SARS-CoV-2 that prevents infection presents as a hope to reduce the infection rate and inhibit the deterioration of the pandemic.

Vaccines include an inactivated vaccine, attenuated vaccine, subunit vaccine such as a protein vaccine and polypeptide vaccine, and nucleic acid vaccine such as DNA vaccine and RNA vaccine. Polypeptide vaccine is a vaccine prepared with chemical synthesis technology according to the amino acid sequence of a known or predicted antigenic epitope in an antigen gene of pathogen. As polypeptide vaccine is completely synthetic, there is no problem of virulence recovery or incomplete inactivation, thus especially suitable for some microbial pathogens that cannot obtain a sufficient amount of antigens through their in vitro culture. Compared with vaccines of other technical routes, polypeptide vaccine of virus epitope is of the following advantages: more suitable for dealing with virus variation: meeting the requirements of rapid and efficient production and reducing the cost of vaccine preparation: no complete virus structure in such a vaccine and correspondingly high safety: allowing various polypeptides derived from different antigens to be combined in one vector; and enabling synthetic antigen polypeptide to be constructed correspondingly for complexly discontinuous natural epitopes.

Although polypeptide vaccines have many advantages, there are still some technical bottlenecks, and, among them, the most important problems are of small molecular weight of polypeptide, low immunogenicity and poor immune response. It is the first key point for the development of polypeptide vaccine to select and design immunogen to stimulate protective immune response correctly and effectively in the human body, since not all polypeptide fragments can stimulate immune response.

### SUMMARY

The present disclosure provides in embodiments a polypeptide antigen from the spike protein of SARS-CoV-2, a polypeptide vaccine and use thereof.

In a first aspect, provided in embodiments of the present disclosure is a polypeptide with a sequence as depicted in any one of SEQ ID NOs: 1 to 116.

Further, the polypeptide is of one or more sequence(s) selected from sequences as depicted in SEQ ID NOs: 1 to 15 and SEQ ID NO: 111.

In a second aspect, provided in embodiments of the present disclosure is an epitope with one or more sequence(s) selected from sequences as depicted in SEQ ID NOs: 1 to 116 as shown in Table 1.

Further, the epitope is of one or more sequence(s) selected from sequences as depicted in SEQ ID NOs: 1 to 15 and SEQ ID NO: 111.

In a third aspect, provided in embodiments of the present disclosure is a polypeptide carrier-protein conjugate including the polypeptide according to any embodiment of the first aspect and a carrier-protein conjugated with the polypeptide.

Further, the polypeptide includes one or more of sequences as depicted in SEQ ID NOs: 1 to 15 and SEQ ID NO: 111.

Further, the carrier-protein is selected from a group consisting of bovine serum albumin, ovalbumin, keyhole limpet hemocyanin and casein.

Further, the polypeptide is conjugated with the carrier-protein via a linker.

Further, 5 to 50 polypeptides, preferably 5 to 30 polypeptides are conjugated with each carrier-protein.

In a fourth aspect, provided in embodiments of the present disclosure is an antigen including one or more polypeptide carrier-protein conjugate(s) according to any embodiment of the third aspect.

In a fifth aspect, provided in embodiments of the present disclosure is a kit for detecting an antibody against coronavirus, where the kit includes the polypeptide according to any embodiment of the first aspect, the epitope according to any embodiment of the second aspect, or the antigen according to any embodiment of the fourth aspect.

Further, the antigen is a precoating antigen.

Further, the precoating antigen coats a solid substrate.

Further, the solid substrate includes an ELISA plate, a membrane or a microsphere.

Further, the membrane includes a nitrocellulose membrane, a glass fiber membrane or a nylon membrane.

Further, the membrane is further coated with a positive reference sequentially arranged with the polypeptide in order of detection.

Further, the kit further includes at least one of the followings: (i) an enzyme-linked secondary antibody, more preferably the enzyme-linked secondary antibody is an HRP-linked secondary antibody: (ii) a colloidal-gold conjugate pad, coated with the polypeptide labeled with the colloidal-gold and the positive reference labeled with the colloidal-gold; and (iii) a control band, coated with a fluorescent-labeled microsphere, the microsphere is loaded with a specific binding material of the positive reference.

Further, the positive reference is selected from murine immunoglobulin, human immunoglobulin, goat immunoglobulin and rabbit immunoglobulin, and correspondingly, the specific binding material of the positive reference is selected from anti-murine immunoglobulin, anti-human immunoglobulin, anti-goat immunoglobulin and anti-rabbit immunoglobulin.

In a sixth aspect, provided in embodiments of the present disclosure is use of the polypeptide according to any embodiment of the first aspect or the epitope according to any embodiment of the second aspect in the manufacture of a medicine for treating a disease caused by coronavirus.

Further, the coronavirus is SARS-CoV-2.

Further, the medicine is an antibody or vaccine.

Further, the vaccine is a polypeptide vaccine or gene vaccine.

In a seventh aspect, provided in embodiments of the present disclosure is a medicine of an antibody or vaccine, where the antibody is obtained by immunizing an animal via the antigen according to any embodiment of the fourth aspect; and the vaccine is a polypeptide vaccine containing the polypeptide according to any embodiment of the first aspect or a gene vaccine containing a nucleic acid encoding the polypeptide according to any embodiment of the first aspect.

Further, the antibody is a neutralizing antibody.

Further, the polypeptide includes one or more of sequences as depicted in SEQ ID NOs: 1 to 15 and SEQ ID NO: 111, and preferably, the polypeptide includes one or more of sequences as depicted in SEQ ID NO: 5, SEQ ID NO: 6, SEQ ID NO: 8, SEQ ID NO: 11, SEQ ID NO: 13 and SEQ ID NO: 14.

In an eighth aspect, provided in embodiments of the present disclosure is a polypeptide composition containing at least two polypeptides selected from sequences as depicted in SEQ ID NOs: 1 to 116.

Further, the polypeptide composition at least contains a polypeptide as depicted in any one of SEQ ID NOs: 1 to 15 and SEQ ID NO: 111.

Further, the polypeptide composition at least contains a polypeptide as depicted in any one of SEQ ID NO: 5, SEQ ID NO: 6, SEQ ID NO: 8, SEQ ID NO: 11, SEQ ID NO: 13 and SEQ ID NO: 14.

In a ninth aspect, provided in embodiments of the present disclosure is a polypeptide vaccine, including one or more of polypeptides as depicted in SEQ ID NOs: 1 to 116.

Further, the polypeptide is at least of a sequence as depicted in any one of SEQ ID NOs: 1 to 15 and SEQ ID NO: 111.

Further, the polypeptide vaccine at least includes a polypeptide as depicted in any one of SEQ ID NO: 5, SEQ ID NO: 6, SEQ ID NO: 8, SEQ ID NO: 11, SEQ ID NO: 13 and SEQ ID NO: 14.

Further, the polypeptide vaccine includes a plurality of polypeptides presented in series or in parallel.

Further, at least one polypeptide in the polypeptide vaccine is repeated for once to 10 times, preferably once to 6 times, more preferably twice to eight times and most preferably 3 to 6 times in series or in parallel.

Further, the plurality of polypeptides are linked in series or in parallel via a linking arm therebetween.

Further, the linking arm for series linking includes Gly, Lys, AEA, Ava, ANP, beta-Ala, GAB or PEG.

Further, the linking arm for parallel linking is one or more selected from the group consisting of Lys, Om, 2,3-diaminopropionic acid, 2,4-diaminobutyric acid, 2,7-diaminoheptanoic acid, 2,8-diaminooctanoic acid, Map-Lys(Map), Map-Orn(Map), Map-2,3-diaminopropionic acid(Map), Map-2,4-diaminobutyric acid(Map), Map-2,7-diaminoheptanoic acid(Map), Map-2,8-diaminooctanoic acid(Map), Map-Lys(Map)-Lys(Map-Lys(Map)), Map-Lys(Map)-Orn(Map-Lys(Map)), Map-Lys(Map)-2,3-diaminopropionic acid(Map-Lys(Map)), Map-Lys(Map)-2,4-diaminobutyric acid(Map-Lys(Map)), Map-Lys(Map)-2,7-diaminoheptanoic acid(Map-Lys(Map)) and Map-Lys(Map)-2,8-diaminooctanoic acid(Map-Lys(Map)), where Map refers to maleimidocaproic acid.

In a tenth aspect, provided in embodiments of the present disclosure is use of the polypeptide carrier-protein conjugate according to any embodiment of the third aspect or the antigen according to any embodiment of the fourth aspect in the manufacture of a vaccine for treating a disease caused by coronavirus.

Further, the coronavirus is SARS-CoV-2. Preferably, the vaccine includes a polypeptide as depicted in any one of SEQ ID NOs: 1 to 15 and SEQ ID NO: 111.

Further, the vaccine at least includes a polypeptide as depicted in any one of SEQ ID NO: 5, SEQ ID NO: 6, SEQ ID NO: 8, SEQ ID NO: 11, SEQ ID NO: 13 and SEQ ID NO: 14.

In an eleventh aspect, provided in embodiments of the present disclosure is a nucleic acid vaccine including a nucleic acid encoding the polypeptide according to any embodiment in the first aspect or the polypeptide composition according to any embodiment in the eighth aspect.

Further, the nucleic acid vaccine is a DNA vaccine or RNA vaccine.

Further, the RNA vaccine is an mRNA vaccine.

In a twelfth aspect, provided in embodiments of the present disclosure is a recombinant protein vaccine including one or more of polypeptides as depicted in SEQ ID NOs: 1 to 116.

Further, the recombinant protein vaccine includes one or more of polypeptides as depicted in SEQ ID NOs: 1 to 15 and SEQ ID NO: 111.

Further, the recombinant protein vaccine is recombined by (i) one or more of polypeptides as depicted in SEQ ID NOs: 1 to 15 and SEQ ID NO: 111; and (ii) 4 to 6 of His or 4 of Gly and 1 of Ser.

Further, the recombinant protein vaccine is recombined by (i) one or more of polypeptides as depicted in SEQ ID NO: 5, SEQ ID NO: 6, SEQ ID NO: 8, SEQ ID NO: 11, SEQ ID NO: 13 and SEQ ID NO: 14; and (ii) 4 to 6 of His or 4 of Gly and 1 of Ser.

Embodiments of the present disclosure are of the following advantages:
a. The polypeptide provided in embodiments of the present disclosure could be used for preparing the related detection reagents such as an antigen, antibody and kit, and the related vaccines such as a polypeptide vaccine, nucleic acid vaccine and recombinant protein vaccine, thus providing a more powerful approach to prevent and control the infection and epidemic of such a virus;
b. The polypeptide provided in embodiments of the present disclosure could stimulate the production of antibodies that bind to the spike protein, as well as antibodies that bind to a RBD domain of the spike protein, with a titer for antibody binding more than 10⁴; and
c. The polypeptide provided in embodiments of the present disclosure stimulate the production of immune serum in a rabbit which presents a certain block to the binding of the angiotensin-converting enzyme 2 (ACE2) and the receptor binding domain (RBD).

### BRIEF DESCRIPTION OF THE DRAWINGS

The above and/or additional aspects and advantages of embodiments of the present disclosure will become apparent and more readily appreciated from the following descriptions made with reference to the drawings. The same or similar elements are denoted by like reference numerals throughout the drawings. It should be understood that embodiments described herein with reference to drawings are explanatory, and components and elements are not necessarily drawn to scale.

FIG. 1 shows purity detection results of polypeptide antigens according to embodiments of the present disclosure, where panel A shows the detection results of a polypeptide antigen with an amino acid sequence as depicted in SEQ ID NO: 1: panel B shows the detection results of a polypeptide antigen with an amino acid sequence as depicted in SEQ ID NO: 6: panel C shows the detection results of a polypeptide antigen with an amino acid sequence as depicted in SEQ ID NO: 8; and panel D shows the detection results of a polypeptide antigen with an amino acid sequence as depicted in SEQ ID NO: 13.

FIG. 2 shows line charts illustrating results of competitive binding assay on immune serum stimulated by a polypeptide antigen mixture according to embodiments of the present disclosure, where chart A shows the results of rabbit antiserum and a coating of RBD to competitively binding to ACE2-Fc; and chart B shows the results of APN01 and the coating of RBD to competitively binding to ACE2-Fc, according to Example 3.

### DETAILED DESCRIPTION

Reference will now be made in detail to embodiments of the present disclosure further, with specific examples therewith. It should be understood that the examples described herein are explanatory; illustrative, and used to generally understand the present disclosure. The embodiments shall not be construed to limit the present disclosure. Any approach realized based on the above contents of the present disclosure is covered within the scope intended to be protected by the present disclosure.

Unless specified otherwise, materials and reagents involved in the following embodiments are commercially available or could be prepared according to known methods. Experimental methods that are not specified with conditions in embodiments below; are generally performed in accordance with the regular conditions such as the conditions described in Li Yongnian, Immunological Laboratory Guide (Science Press, 2018); and Yu Ping, Immunological Experiment (Huazhong University of Science & Technology Press, 2012), or in accordance with conditions provided by the manufacturers.

### Terms and Illustrations

### Coronavirus

The term “coronavirus” herein refers to a single-stranded positive-sense RNA virus, belonging to Nidovirales Coronaviridae subfamily Orthocoronavirinae. The coronavirus can infect humans, bats, pigs, mice, cattle, horses, goats, monkeys and other species. There are seven coronaviruses known to infect humans (HCoV), including the Middle East respiratory syndrome-related coronavirus (MERSr-CoV) and severe acute respiratory syndrome-related coronavirus (SARSr-CoV).

In specific embodiments, the “coronavirus” herein refers to the severe acute respiratory syndrome coronavirus (SARS-CoV), the Middle East respiratory syndrome coronavirus (MERS-CoV) or SARS-CoV-2. In preferable embodiments, the “coronavirus” herein refers to the SARS-CoV or 2019 novel coronavirus (2019-nCoV), more preferably, to the 2019-nCoV.

This newly isolated coronavirus, a novel betacoronavirus and named as “2019-nCoV” by the WHO, is the seventh coronavirus able to infect humans. In order to continuously adapt to the host, this novel coronavirus constantly mutates at nucleotide sites during its replication, potentially causing some variants that affect the transmission, pathogenicity and immunogenicity of the virus. At present, there are mainly five kinds of 2019-nCoV variants, namely, Alpha, Beta, Gamma, Delta and Lambda. Currently, responses to SARS-CoV-2 mainly include controlling the spread of the virus through preventive measures, closely monitoring the epidemic situation, isolating suspected cases for observation, and injecting vaccines. There is no specific treatment for coronavirus so far, merely adopting symptomatic and supportive treatment mainly.

SARS-CoV-2 enters cells by binding to the cell surface receptor ACE2 of human through the spike(S) protein on the virus surface. S protein is composed of a long extra-membrane domain, transmembrane domain and intra-membrane domain, belonging to Class I viral fusion protein. S proteins of different coronavirus significantly differs from each other most on whether they are cleaved by host proteases during viral assembly and release. Mature S proteins are usually cleaved into two subunits, S1 and S2, by host proteases such as cysteine proteases, trypsin, etc. The S1 subunit may be further divided into two relatively independent domains, namely an N-terminal domain and a C-terminal domain, and the S1 subunit contains a receptor binding domain (RBD), most of which are located in the C-terminal domain of the S protein of coronavirus. The S2 subunit anchors to the membrane by the transmembrane domain, which contains essential elements for membrane fusion, including an intrinsic fusion peptide (FP), two heptad repeats (HR) each having 7 amino acids, a juxamembrain domain (JMD), the transmembrane domain (TMD), and a cytoplasmic domain (CD) with a length of about 40 amino acids at the C-terminal. The two HR, HR1 and HR2, also called HR-N and HR-C according to their position, are separated by a helix structure therebetween formed by about 140 amino acids. When RBD binds to the receptor, the S2 subunit changes its conformation by inserting the FP into the host cell membrane, where HR1 and HR2 each form a triple helix structure, and the two triple-helix structure are arranged anti-parallelly, thus forming a six-helix bundle (6HB), which together form a fusion core finally leading to the fusion of virus membrane and cell membrane. Therefore, blocking RBD recognition to the host cells and the fusion of the S2 subunit with cell membrane can effectively inhibit virus invasion.

The S protein is of an ideal antigen because of its important function. However. SARS-CoV-2 concerns an RNA virus, vaccines of which often produce side effects, such as antibody dependent enhancement (ADE). These side effects are often caused by components within vaccines stimulating an immune response that is not protective.

The term “(antigen) epitope” refers to an antigen that stimulates the immune system of the body to generate specific immune response and specifically binds to the corresponding immune response products such as antibodies or sensitized lymphocytes in vivo or in vitro. Antigen epitope, also known as antigenic determinant, is specific chemical groups with certain composition and structure on the surface or other parts of an antigen and determines antigenic specificity. During immune response, epitopes recognized by TCR and BCR have different characteristics, which are called T cell epitopes and B cell epitopes respectively. T cell epitopes are generally not located on the surface of antigens and required to be processed into small molecular polypeptides by antigen presenting cells and bound to MHC molecules, for recognition by TCR. That is, T cells can only recognize the processed epitopes. In contrast, B cell epitopes may present on the surface of antigens and can be recognized directly by B cells without processing. In embodiments of the present disclosure, the term “epitope” refers to one or more peptide(s) that, with prediction or selection, is (are) able to bind to an antigen receptor on the surface of a corresponding lymphocyte, thereby activating the lymphocyte to generate an immune response, and able to specifically bind to a corresponding antibody or sensitized lymphocyte to exert immune effects, where the antibody is a specific antibody.

The term “polypeptide” herein refers to any peptide that, with prediction or selection, is able to specifically bind to an antibody or sensitized lymphocyte.

The term “polypeptide carrier-protein conjugate” herein refers to an antigen formed by conjugating a polypeptide with a carrier-protein, where one or more polypeptide(s) may be conjugated with each carrier-protein. When more than one polypeptides are conjugated, these polypeptides may have the same or different sequences thereamong. According to the differences in physicochemical properties of specific conjugated polypeptides, the types of specific carrier proteins and methods for conjugation, the number of polypeptides conjugated with each carrier protein varies. In embodiments of the present disclosure, preferably 3 to 50 polypeptides, more preferably 3 to 45, 5 to 40, 5 to 35, 5 to 30, 8 to 30, 10 to 30, 12 to 30 or 15 to 30 polypeptides: or, more preferably 6 to 36, 8 to 32, 10 to 28, 10 to 26, 10 to 24, 10 to 22, 10 to 20, 10 to 18, 10 to 16 or 10 to 15 polypeptides are conjugated with each carrier protein.

The term “vaccine” is generally of both immunogenicity and reactogenicity. Immunogenicity refers to the ability to stimulate the body to generate an immune response, that is, to stimulate specific immune cells of the body into activation, proliferation and differentiation, and finally produce immune effector substances such as specific antibodies or sensitized lymphocytes, while reactogenicity refers to the ability to specifically bind to antibodies or sensitized lymphocytes induced by the vaccine.

For the term “polypeptide vaccine”, in order to improve immunogenicity of a polypeptide so as to stimulate the body to produce specific antibodies or sensitized lymphocytes, the polypeptide antigen is usually combined with an adjuvant for compatible immunization. Adjuvants that are commonly used include: aluminum hydroxide, Corynebacteriuum parvm, lipopolysaccharide, cytokines or alum, etc., and complete Freund's adjuvant and incomplete Freund's adjuvant are the most common adjuvants in animal immunization.

Unless otherwise defined or clearly specified by the context, all technical and scientific terms in the disclosure are of the same meaning as commonly understood by one of ordinary skill in the art to which the disclosure pertains.

Embodiments of the present disclosure provide a polypeptide antigen from the spike protein of SARS-CoV-2, a polypeptide vaccine and use thereof. The polypeptide is selected from any one of sequences as depicted in SEQ ID NOs: 1 to 116, as shown in Table 1.

TABLE 1

| | | | | Accession No. |
|---|---|---|---|---|
| | | Positions | Protein the | of the protein |
| SEQ ID | | of the | polypeptide | the polypeptide |
| NO: | Polypeptide | polypeptide | derived from | derived from |
| 1 | HKNNKSWMESEFRVYSSA | 146-163 | S1 | EPI_ISL_402124 |
| 2 | KHTPINLVRDLPQGFS | 206-221 | S1 | EPI_ISL_402124 |
| 3 | HRSYLTPGDSSSG | 245-257 | S1 | EPI_ISL_402124 |
| 4 | YGVSPTKLNDLC | 380-391 | RBD | EPI_ISL_402124 |
| 5 | RGDEVRQIAPGQTGKIADYNYKL | 403-425 | RBD | EPI_ISL_402124 |
| 6 | APGQTGKIADYNYKLPDDFTGC | 411-432 | RBD | EPI_ISL_402124 |
| 7 | SKVGGNYNYLYRLFRKSNLKPFERDISTE | 443-471 | RBD | EPI_ISL_402124 |
| 8 | KSNLKPFERDISTEIYQAGSTPC | 458-480 | RBD | EPI_ISL_402124 |
| 9 | CYFPLQSYGFQPTNGVGYQPYR | 488-509 | RBD | EPI_ISL_402124 |
| 10 | CGPKKSTNLVKNK | 525-537 | RBD | EPI_ISL_402124 |
| 11 | TTDAVRDPQTLE | 572-583 | S1 | EPI_ISL_402124 |
| 12 | IKDFGGFNFSQILPDPSKPSKRS | 794-816 | S2 | EPI_ISL_402124 |
| 13 | YDPLQPELDSFKEELDKYFK | 1138-1157 | S2 | EPI_ISL_402124 |
| 14 | KNHTSPDVDLGDIS | 1157-1170 | S2 | EPI_ISL_402124 |
| 15 | CKFDEDDSEPVLK | 1254-1266 | S2 | EPI_ISL_402124 |
| 16 | GVYYHKNNKSWM | 142-153 | S1 | EPI_ISL_402124 |
| 17 | GVYYHKNNKSWMESEFRV | 142-159 | S1 | EPI_ISL_402124 |
| 18 | GVYYHKNNKSWMESEFRVYSSANNCTF | 142-168 | S1 | EPI_ISL_402124 |
| 19 | VYYHKNNKSWMESEFRVY | 143-160 | S1 | EPI_ISL_402124 |
| 20 | YHKNNKSWMESEFRV | 145-159 | S1 | EPI_ISL_402124 |
| 21 | HKNNKSWMESEFRVYSSANNCTF | 146-168 | S1 | EPI_ISL_402124 |
| 22 | KSWMESEFRVYSSANNCTFEYVSQPFLMDL | 150-179 | S1 | EPI_ISL_402124 |
| 23 | KIYSKHTPINLVRDLPQGFS | 202-221 | S1 | EPI_ISL_402124 |
| 24 | KHTPINLVRDLPQGF | 206-220 | S1 | EPI_ISL_402124 |
| 25 | FSALEPLVDLPIGINITR | 220-237 | S1 | EPI_ISL_402124 |
| 26 | FSALEPLVDLPIGINITRFQTLLALH | 220-245 | S1 | EPI_ISL_402124 |
| 27 | KLNDLCFTNVYADSFVIR | 386-403 | RBD | EPI_ISL_402124 |
| 28 | RGDEVRQIAPGQTGKIAD | 403-420 | RBD | EPI_ISL_402124 |
| 29 | RGDEVRQIAPGQTGKIADYN | 403-422 | RBD | EPI_ISL_402124 |
| 30 | RGDEVRQIAPGQTGKIADYNYKLPDD | 403-428 | RBD | EPI_ISL_402124 |
| 31 | VRQIAPGQTGKIAD | 407-420 | RBD | EPI_ISL_402124 |
| 32 | VRQIAPGQTGKIADYNYKLPDDFTGCV | 407-433 | RBD | EPI_ISL_402124 |
| 33 | RQIAPGQTGKIADYNYKL | 408-425 | RBD | EPI_ISL_402124 |
| 34 | RQIAPGQTGKIADYNYKLPDDFTGCV | 408-433 | RBD | EPI_ISL_402124 |
| 35 | APGQTGKIADYNYKLPDDFTGCV | 411-433 | RBD | EPI_ISL_402124 |
| 36 | GNYNYLYRLFRKSNLKPFERDIS | 447-469 | RBD | EPI_ISL_402124 |
| 37 | GNYNYLYRLFRKSNLKPFERDISTEI | 447-472 | RBD | EPI_ISL_402124 |
| 38 | NYNYLYRLFRKSNLKPFER | 448-466 | RBD | EPI_ISL_402124 |
| 39 | YRLFRKSNLKPFERDISTEI | 453-472 | RBD | EPI_ISL_402124 |
| 40 | KSNLKPFERDISTEI | 458-472 | RBD | EPI_ISL_402124 |
| 41 | KSNLKPFERDISTEIYQAGSTPCNGV | 458-483 | RBD | EPI_ISL_402124 |
| 42 | VEGFNCYFPLQSYGFQPTNGVGYQPYR | 483-509 | RBD | EPI_ISL_402124 |
| 43 | NCYFPLQSYGFQPTNGV | 487-503 | RBD | EPI_ISL_402124 |
| 44 | CYFPLQSYGFQPTNGVGYQPYRVVVLSF | 488-515 | RBD | EPI_ISL_402124 |
| 45 | RDIADTTDAVRDPQTLEI | 567-584 | S1 | EPI_ISL_402124 |
| 46 | VEQDKNTQEVFAQVKQIYKTPPIKDF | 772-797 | S2 | EPI_ISL_402124 |
| 47 | IYKTPPIKDFGGFNFSQILPDPSKPSK | 788-814 | S2 | EPI_ISL_402124 |
| 48 | IYKTPPIKDFGGFNFSQILPDPSKPSKRS | 788-816 | S2 | EPI_ISL_402124 |
| 49 | TVYDPLQPELDSFK | 1136-1149 | S2 | EPI_ISL_402124 |
| 50 | TVYDPLQPELDSFKEELDKYFK | 1136-1157 | S2 | EPI_ISL_402124 |
| 51 | TVYDPLQPELDSFKEELDKYFKNH | 1136-1159 | S2 | EPI_ISL_402124 |
| 52 | TVYDPLQPELDSFKEELDKYFKNHTSPDVDL | 1136-1170 | S2 | EPI_ISL_402124 |
| | GDIS | | | |
| 53 | VYDPLQPELDSF | 1137-1148 | S2 | EPI_ISL_402124 |
| 54 | VYDPLQPELDSFKEELDKYFK | 1137-1157 | S2 | EPI_ISL_402124 |
| 55 | YDPLQPELDSFKE | 1137-1159 | S2 | EPI_ISL_402124 |
| 56 | YDPLQPELDSFKE | 1138-1150 | S2 | EPI_ISL_402124 |
| 57 | YDPLQPELDSFKEELDKYFKN | 1138-1158 | S2 | EPI_ISL_402124 |
| 58 | YDPLQPELDSFKEELDKYFKNH | 1138-1159 | S2 | EPI_ISL_402124 |
| 59 | YDPLQPELDSFKEELDKYFKNHTSPDVDLGD | 1138-1170 | S2 | EPI_ISL_402124 |
| | IS | | | |
| 60 | DPLQPELDSFKEELDKYFK | 1139-1157 | S2 | EPI_ISL_402124 |
| 61 | DPLQPELDSFKEELDKYFKN | 1139-1158 | S2 | EPI_ISL_402124 |
| 62 | PLQPELDSFKEELDKYFK | 1140-1157 | S2 | EPI_ISL_402124 |
| 63 | PLQPELDSFKEELDKYFKN | 1140-1158 | S2 | EPI_ISL_402124 |
| 64 | PLQPELDSFKEELDKYFKNHTSPDVDLGDIS | 1140-1170 | S2 | EPI_ISL_402124 |
| 65 | EELDKYFKNHTSPDVDLGDIS | 1150-1170 | S2 | EPI_ISL_402124 |
| 66 | EELDKYFKNHTSPDVDLGDISGINA | 1150-1174 | S2 | EPI_ISL_402124 |
| 67 | KNHTSPDVDLGDISGINA | 1157-1174 | S2 | EPI_ISL_402124 |
| 68 | FDEDDSEPVL | 1256-1265 | S2 | EPI_ISL_402124 |
| 69 | (Delta)HKNNKSWMESGFRVYSSA | 146-163 | S1 | EPI_ISL_3791379 |
| 70 | (Beta)KHTPINLVRGLPQGFS | 206-221# | S1 | EPI_ISL_5797435 |
| 71 | (Beta)RGDEVRQIAPGQTGNIADYNYKL | 403-425 | RBD | EPI_ISL_5797435 |
| 72 | (Gamma)RGDEVRQIAPGQTGTIADYNYKL | 403-425 | RBD | EPI_ISL_3933507 |
| 73 | (Beta)APGQTGNIADYNYKLPDDFTGC | 411-432 | RBD | EPI_ISL_5797435 |
| 74 | (Gamma)APGQTGTIADYNYKLPDDFTGC | 411-432 | RBD | EPI_ISL_3933507 |
| 75 | (Delta)SKVGGNYNYRYRLFRKSNLKPFERDIS | 443-471 | RBD | EPI_ISL_3791379 |
| | TE | | | |
| 76 | (Delta)KSNLKPFERDISTEIYQAGSKPC | 458-480 | RBD | EPI_ISL_3791379 |
| 77 | (Alpha\Beta\Gamma)CYFPLQSYGFQPTYGVG | 488-509 | RBD | EPI_ISL_5830465 |
| | YQPYR | | | |
| 78 | (Delta)GVYYHKNNKSWMESGFRV | 142-159 | S1 | EPI_ISL_3791379 |
| 79 | (Delta)GVYYHKNNKSWMESGFRVYSSANNC | 142-168 | S1 | EPI_ISL_3791379 |
| | TF | | | |
| 80 | (Delta)VYYHKNNKSWMESGFRVY | 143-160 | S1 | EPI_ISL_3791379 |
| 81 | (Delta)YHKNNKSWMESGFRV | 145-159 | S1 | EPI_ISL_3791379 |
| 82 | (Delta)HKNNKSWMESGFRVYSSANNCTF | 146-168 | S1 | EPI_ISL_3791379 |
| 83 | (Delta)KSWMESGFRVYSSANNCTFEYVSQPF | 150-179 | S1 | EPI_ISL_3791379 |
| | LMDL | | | |
| 84 | (Beta)KIYSKHTPINLVRGLPQGFS | 202-221 | S1 | EPI_ISL_5797435 |
| 85 | (Beta)KHTPINLVRGLPQGF | 206-220 | S1 | EPI_ISL_5797435 |
| 86 | (Beta)RGDEVRQIAPGQTGNIAD | 403-420 | RBD | EPI_ISL_5797435 |
| 87 | (Gamma)RGDEVRQIAPGQTGTIAD | 403-420 | RBD | EPI_ISL_3933507 |
| 88 | (Beta)RGDEVRQIAPGQTGNIADYN | 403-422 | RBD | EPI_ISL_5797435 |
| 89 | (Gamma)RGDEVRQIAPGQTGTIADYN | 403-422 | RBD | EPI_ISL_3933507 |
| 90 | (Beta)RGDEVRQIAPGQTGNIADYNYKLPDD | 403-428 | RBD | EPI_ISL_5797435 |
| 91 | (Gamma)RGDEVRQIAPGQTGTIADYNYKLPD | 403-428 | RBD | EPI_ISL_3933507 |
| | D | | | |
| 92 | (Beta)VRQIAPGQTGNIAD | 407-420 | RBD | EPI_ISL_5797435 |
| 93 | (Gamma)VRQIAPGQTGTIAD | 407-420 | RBD | EPI_ISL_3933507 |
| 94 | (Beta)VRQIAPGQTGNIADYNYKLPDDFTGCV | 407-433 | RBD | EPI_ISL_5797435 |
| 95 | (Gamma)VRQIAPGQTGTIADYNYKLPDDFTG | 407-433 | RBD | EPI_ISL_3933507 |
| | CV | | | |
| 96 | (Beta)RQIAPGQTGNIADYNYKL | 408-425 | RBD | EPI_ISL_5797435 |
| 97 | (Gamma)RQIAPGQTGTIADYNYKL | 408-425 | RBD | EPI_ISL_3933507 |
| 98 | (Beta)RQIAPGQTGNIADYNYKLPDDFTGCV | 408-433 | RBD | EPI_ISL_5797435 |
| 99 | (Gamma)RQIAPGQTGTIADYNYKLPDDFTGC | 408-433 | RBD | EPI_ISL_3933507 |
| | V | | | |
| 100 | (Beta)APGQTGNIADYNYKLPDDFTGCV | 411-433 | RBD | EPI_ISL_5797435 |
| 101 | (Gamma)APGQTGTIADYNYKLPDDFTGCV | 411-433 | RBD | EPI_ISL_3933507 |
| 102 | Delta)GNYNYRYRLFRKSNLKPFERDIS | 447-469 | RBD | EPI_ISL_3791379 |
| 103 | (Delta)GNYNYRYRLFRKSNLKPFERDISTEI | 447-472 | RBD | EPI_ISL_3791379 |
| 104 | (Delta)NYNYRYRLFRKSNLKPFER | 448-466 | RBD | EPI_ISL_3791379 |
| 105 | (Delta)KSNLKPFERDISTEIYQAGSKPCNGV | 458-483 | RBD | EPI_ISL_3791379 |
| 106 | (Delta)DVYYHKNNKSWM | 142-153 | S1 | EPI_ISL_3791379 |
| 107 | (Alpha\Gamma)VKGFNCYFPLQSYGFQPTYGV | 483-509 | RBD | (Alpha)EPI_ISL_ |
| | GYQPYR | | | 5830465 |
| | | | | (Gamma)EPI_ISL_ |
| | | | | 3933507 |
| 108 | (Alpha\Gamma)NCYFPLQSYGFQPTYGV | 487-503 | RBD | (Alpha)EPI_ISL_ |
| | | | | 5830465 |
| | | | | (Gamma)EPI_ISL_ |
| | | | | 3933507 |
| 109 | (Alpha\Gamma)CYFPLQSYGFQPTYGVGYQPY | 488-515 | RBD | (Alpha)EPI_ISL_ |
| | RVVVLSF | | | 5830465 |
| | | | | (Gamma)EPI_ISL_ |
| | | | | 3933507 |
| 110 | (Alpha)RDIDDTTDAVRDPQTLEI | 567-584 | S1 | EPI_ISL_5830465 |
| 111 | NYLYRLFRKSNLKPFERDISTE | 450-471 | RBD | EPI_ISL_402124 |
| 112 | NYNYLYRLFRKSNLKPFERDISTE | 448-471 | RBD | EPI_ISL_402124 |
| 113 | GGNYNYLYRLFRKSNLKPFERDISTE | 446-471 | RBD | EPI_ISL_402124 |
| 114 | (Delta)NYRYRLFRKSNLKPFERDISTE | 450-471 | RBD | EPI_ISL_3791379 |
| 115 | (Delta)NYNYRYRLFRKSNLKPFERDISTE | 448-471 | RBD | EPI_ISL_3791379 |
| 116 | (Delta)GGNYNYRYRLFRKSNLKPFERDISTE | 446-471 | RBD | EPI_ISL_3791379 |

### Example 1: Design of Polypeptide Antigen

An epitope screening are usually based on a target protein sequence for prediction with access bioinformatics software or selection according to known knowledge. In this Example, 116 polypeptides were designed through immunogenicity analysis and secondary structure and hydrophobicity predictions on the S protein in the whole genome sequence of SARS-CoV-2 (EPI_ISL_402124), for predicting potential surface regions of the S protein, and the sequence lengths and characteristics thereof are shown in Table 1.

In Examples, SEQ ID NOs: 1 to 15, 111 and 114 in Table 1 were selected as subsequent vaccine peptides, and the details are shown in Table 2 below.

### Example 2: Chemosynthesis of the Polypeptide Antigen Selected Above and Preparation of Immunogen

### 2.1 Polypeptide Synthesis

Polypeptides were individually synthesized from a C-terminal to an N-terminal thereof to obtain a peptide resin adopting the organic chemical solid phase synthesis method with Fmoc protected amino acids and a resin as solid phase by using three-channel polypeptide automatic synthesizer (CS360, American CS company), and the polypeptides were cut from the resin with TFA method, followed by preliminary extraction so as to obtain crude products.

### 2.2 Polypeptide Purification

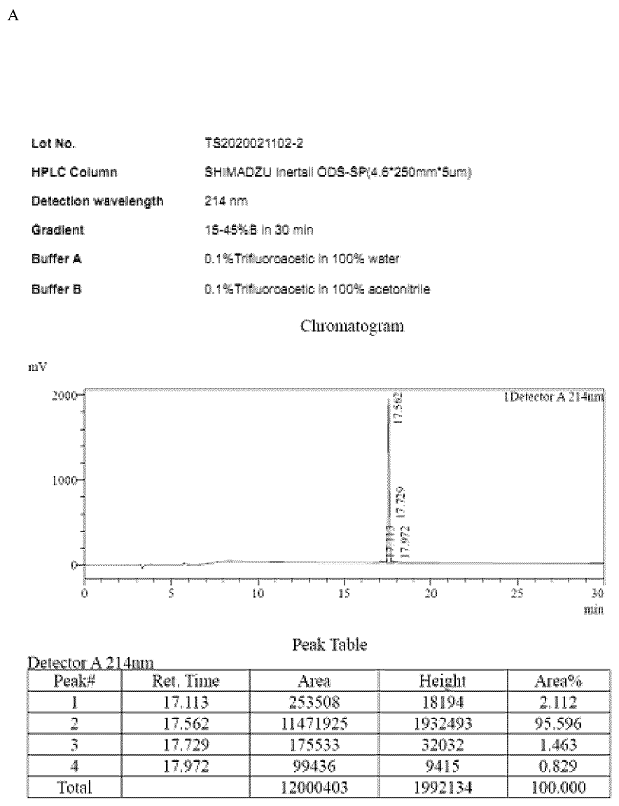

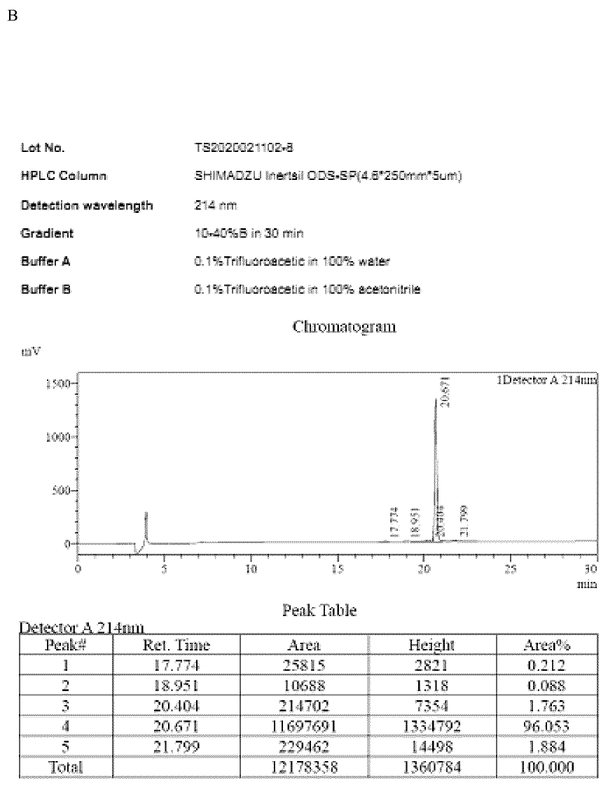

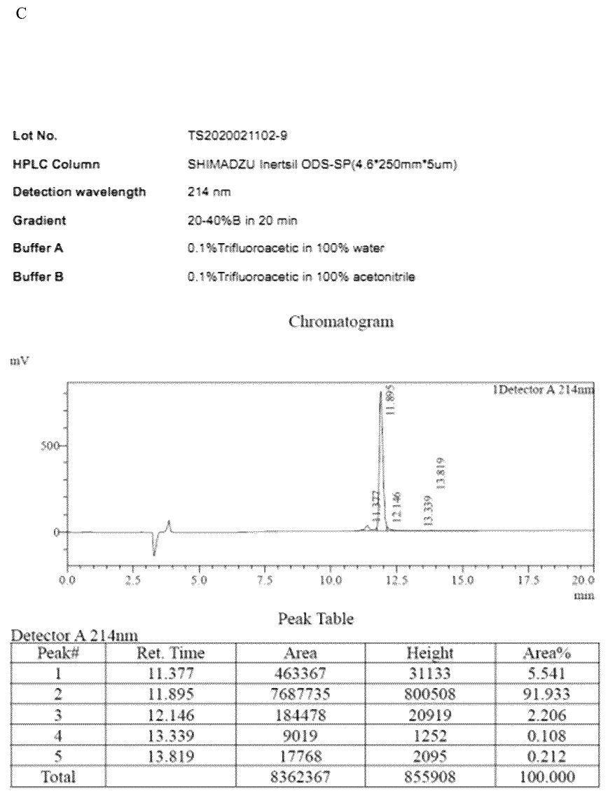

Polypeptides were chromatographed and purified with C18 reversed-phase chromatographic columns and high-performance liquid chromatograph (Waters, U.S.), and then subject to frozen-drying. Respective purity of these 16 synthetic polypeptides is more than 90%. The results of purification are shown in FIG. 1.

### 2.3 Preparation of Immunogen

Keyhole limpet hemocyanin (KLH) is a free blue respiratory pigment discovered in the hemolymph of mollusks and arthropods (spiders and beetles). KLH is of high immunogenicity and is the most commonly used carrier protein. For conjugating the polypeptide with KLH as the carrier protein, 10 mg of each of the purified polypeptides and 20 mg of KLH were subject to condensation under the catalysis of a condensing agent, so as to obtain corresponding polypeptide-KLH conjugates (polypeptide-KLH).

Specific steps for conjugating each polypeptide with KLH were as follows.

1) 20 Mg of KLH were dissolved in PBS (pH 7) to a final concentration of 10 mg/ml, then added with m-maleimidobenzoyl-N-hydroxysuccinimide ester (MBS, Thermo Fisher) as a coupling reagent, to react at room temperature for 1 h thus to form a KLH-MBS complex. After that, the free coupling reagent therein was removed by dialysis with PBS.

2) 10 Mg of each polypeptide was dissolved in PBS to a concentration of 10 mg/ml, where the polypeptide without Cys was introduced with Cys at its N-terminal or C-terminal.

3) Each polypeptide solution was mixed with the prepared KLH-MBS and reacted for 2 h at room temperature.

4) Each reaction mixture was subject to dialysis with PBS, thereby obtaining the immunogen of KLH-polypeptide after stopping the reaction.

5) With protein quantification, respective immunogen was of a concentration about 5 mg/ml, in which the polypeptide was about 0.5 mg/ml.

### Example 3: Animal Immunization

Animals, i.e. New Zealand white rabbits purchased from Qingdao Kangda Biotechnology Co., Ltd, were the first class animals passing quarantine without pathogenic bacteria, for each weighed about 1.5 kg. After one week's observation, rabbits were healthy and lively, with shiny fur and normal diet. These rabbits were then performed with immunity, and the specific immunity treatments were as follows.

Each polypeptide-KLH prepared in Example 2 was mixed with complete Freund's adjuvant (basic immunization) or incomplete Freund's adjuvant (booster immunization), fully emulsified, and injected intradermally into the back of rabbits at multiple points, with the total amount injected into per rabbit not exceeding 1.5 ml, containing about 1 mg of KLH-polypeptide with about 100 μg of polypeptide epitope therein. Each polypeptide antigen was injected into three rabbits (as immunological replications) once every 3 weeks for 4 times, for each rabbit. On the 10th day after the fourth immunization, blood was collected from ear vein of the animals, for separating serum to perform determination. Additionally, a group treated with KLH alone without conjugated polypeptide antigen and a group treated with adjuvant alone were set as control groups, where animals in the group treated with KLH alone were injected with KLH mixed with complete Freund's adjuvant with respective amount equal to that in the polypeptide-KLH group, and the group treated with adjuvant alone was injected with complete Freund's adjuvant with its original concentration, with an equal amount to the final solution injected in the polypeptide-KLH group. And the groups treated with KLH alone and adjuvant alone were performed with the same administration manner and frequency as the polypeptide-KLH group.

### Example 4: Antibody Binding Assay on Polypeptides

Antibody binding titer of each polypeptide, prepared by the organic chemical solid phase synthesis and without conjugation with KLH, was determined through an ELISA plate coated with such a polypeptide. Specific steps were as follows.

1) Preparing coating solution: 100 μl of a polypeptide solution (2 mg/ml) was mixed evenly with 100 ml of carbonate buffer solution at 0.05 M, to obtain a coating solution with concentration of 2 μg/ml. And blank control and negative control were set.

2) To each well of the ELISA plate. 100 μl of the prepared polypeptide solution were added, reacting at 4° C. overnight. After that, the liquid in the well was discarded.

3) Blocking in ELISA wells: with each well, a blocking reagent of 5% calf serum was fully filled and blocked at 37° C. for 40 min, after removal of bubbles in each well.

4) Washing: for each well, the liquid therein was removed and the wells were fully filled with a washing solution for washing for 3 min with slight shaking. Then, the liquid in the wells was poured off and the plate was patted dry on absorbent paper. This step was performed 3 times.

5) Adding samples: samples for test were introduced into a gradient dilution plate to establish appropriate concentration gradients, such as 1:500, 1:2000, 1:8000, 1:32000, 1:128000, 1:512000, and 1:2048000. The diluted samples were added into the wells of the ELISA plate, with 3 wells for each sample and 100 μl for each well, and incubated at 37° C. for 60 min. Then the wells were washed with the washing solution fully filled for 3 times, with 3 min each time.

6) Adding enzyme-linked antibody: goat anti-rabbit IgG labeled with horseradish peroxidase (ZSGB-BIO) as a secondary antibody, was diluted with PBS (pH 7.4) at a ratio of 1:20000, and 100 μl of the diluted secondary antibody were added into each well for incubating at 37° C. for 60 min. Then the wells were washed with the washing solution fully filled for 3 times, with 3 min each time.

7) Chromogenic reaction: taking tetramethyl benzidine (TMB) as a chromogenic reagent, 100 μl of TMB/H2O2-urea solution were added to each well and incubated at 37° C. for 3-5 minutes in the dark, and then added with 50 μl of stop buffer (sulfuric acid solution at 2 M) for each well to stop the reaction.

8) Detection: the optical density at 450 nm was determined within 20 min after stopping the chromogenic reaction.

The results are shown in Table 2. It can be seen that with immunization to the rabbits by the 16 polypeptide antigens individually, they all produced antibodies against respective polypeptides in serum, with high titers for antibodies more than 10⁵. No titer was detected in the group treated with KLH alone or adjuvant alone.

TABLE 2
Summary of characteristics and immunizing titers of polypeptides

| SEQ | | Titers of | Titers of | Titers of |
|---|---|---|---|---|
| ID | | polypeptides | recombinant S | RBD |
| NO: | Sequence | binding | protein binding | binding |
| 1 | HKNNKSWMESEFRVYSSA | 2048000 | 8000 | 40 |
| 2 | KHTPINLVRDLPQGFS | 2048000+ | 8000 | 20 |
| 3 | HRSYLTPGDSSSG | 512000 | 2000 | 60 |
| 4 | YGVSPTKLNDLC | 2048000 | 8000 | 20000 |
| 5 | RGDEVRQIAPGQTGKIADYNYKL | 2048000 | 2048000 | 320000 |
| 6 | APGQTGKIADYNYKLPDDFTGC | 2048000 | 128000 | 60000 |
| 7 | SKVGGNYNYLYRLFRKSNLKPFERDISTE | 2048000 | 32000 | 35000 |
| 8 | KSNLKPFERDISTEIYQAGSTPC | 2048000 | 2048000 | 150000 |
| 9 | CYFPLQSYGFQPTNGVGYQPYR | 2048000 | 32000 | 1500 |
| 10 | CGPKKSTNLVKNK | 512000 | 2000 | 15000 |
| 11 | TTDAVRDPQTLE | 512000 | 128000 | 80 |
| 12 | IKDFGGFNFSQILPDPSKPSKRS | 2048000 | 32000 | 120 |
| 13 | YDPLQPELDSFKEELDKYFK | 2048000 | 2048000 | <10 |
| 14 | KNHTSPDVDLGDIS | 2048000 | 512000 | <20 |
| 15 | CKFDEDDSEPVLK | 2048000 | 2000 | <20 |
| 111 | NYLYRLFRKSNLKPFERDISTE | 2048000 | 128000 | 35000 |

### Example 5: Antibody Binding Assay on S Protein

The protocol was the same as in Example 4, with a coating concentration of 0.1 μg/well (1 μg/ml) of S protein (Beijing Bioscience Co., Ltd.). The results are shown in Table 2. It can be seen that immunization with each of polypeptides 5 to 9^{#} and 11 to 14^{#} produced antibodies against S protein in the serum at high levels, where the antibody binding titer was above 10⁵ for each of polypeptides 5^{#}, 6^{#}, 8^{#}, 11^{#}, 13^{#} and 14^{#}, and above 10⁴ for each of polypeptides 7^{#}, 9^{#} and 12^{#}.

### Example 6: Antibody Binding Assay on RBD of S Protein

RBD of S protein (Shanghai Huicheng Biotechnology Co., Ltd.), as an antigen, was for coating overnight at 4° C. at concentration of 1 μg/ml. Serum dilutions were started with a dilution ratio of 1:30 and diluted in 10-fold gradients. Incubation time was set to 2 h. HRP-linked goat anti-rabbit antibody as a secondary antibody, was diluted at 1:20000, and incubated for 1 h. After that, absorbance at 450 nm was detected after chromogenic reaction with TMB.

The results are shown in Table 2. It can be seen that immunization with each of polypeptides 4 to 8^{#} and 10^{#} produced antibodies against RBD in the serum at high levels, where the antibody binding titer was above 10⁵ for each of polypeptides 5^{#} and 8^{#}, and above 10⁴ for each of polypeptides 4^{#}, 6^{#}, 7^{#} and 10^{#}, which was consistent with that of RBD of S protein having these polypeptides. Although antibodies in serum against each of polypeptides 11 to 14^{#} had high binding activities to S protein, such antibodies showed no binding activity with RBD, which was consistent with that of RBD without these polypeptides.

### Example 7: Competitive Antigen Binding Assay with Immune Serum

RBD of S protein, as an antigen, was for coating overnight at 4° C. at concentration of 1 μg/ml. Serum antigens of polypeptides 5 to 8^{#} were mixed in equal amount and dilute at 1:4 to obtain a working concentration. Taking the working concentration as a first point, it was diluted with 3-fold gradient. To each well of the coated plate. 50 μl of the diluted serum was added and incubated for 30 min. and then added with 50 μl of ACE-Fc (Beijing Bio-Lab Technology Co., Ltd.) at 0.1 μg/ml and incubated for 1 h. HRP-linked goat anti-human Fc antibody (Abcam, ab6721) as a secondary antibody, was diluted at 1:30000, and incubated for 1 h. After that, absorbance at 450 nm was detected after chromogenic reaction with TMB.

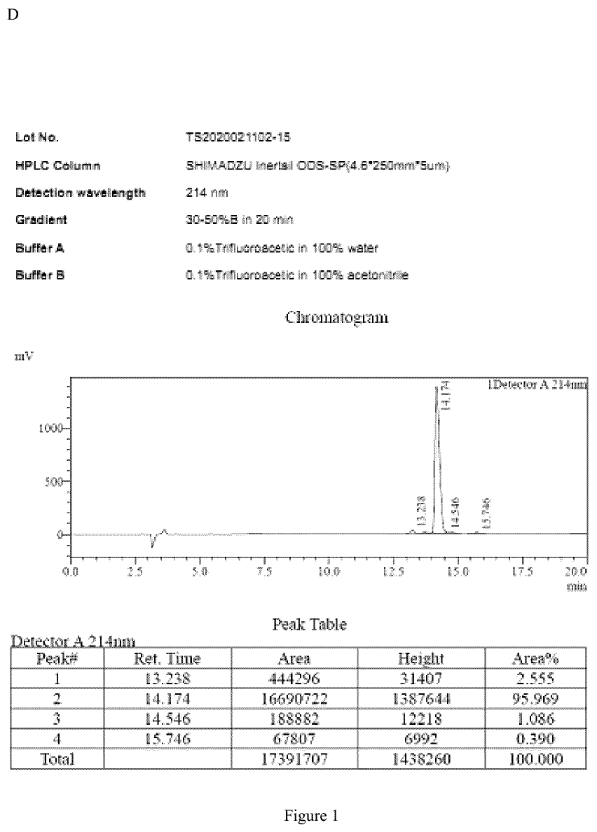

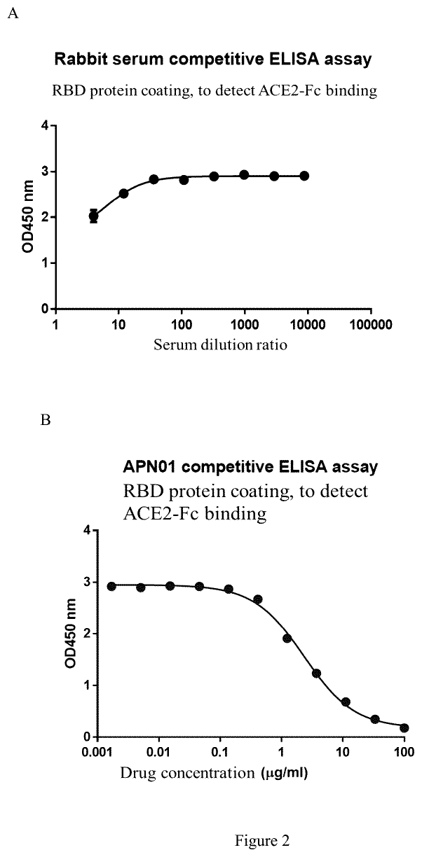

The results are shown in FIG. 2, in which, at dilution ratios of 1:4 and 1:12, it was observed that the mixed rabbit immune serum blocked the binding of ACE2 with RBD to some extent. APN01 was the positive control in parallel experiments.

### Example 8: Preparation of Polypeptide Antigen and Immunocompetence Evaluation Thereof

8.1 Preparation of Polypeptide with Four Branches

8.1.1 Based on an linking arm for parallel linking being one or more selected from the group consisting of Lys, Orn, 2,3-diaminopropionic acid, 2,4-diaminobutyric acid, 2,7-diaminoheptanoic acid and 2,8-diaminooctanoic acid, the linking arm including amino acids which was double Fmoc-protected was firstly introduced into a resin, and the obtained resin may be used to prepare two polypeptides linked in parallel. Further, by introducing the resin above the double Fmoc-protected linking arm including amino acids once more, the obtained resin may be used to prepare four polypeptides linked in parallel, and so on.

Introductions of amino acids and purifications of amino acid sequences were the same as synthesis of the linear polypeptide as described above. A polypeptide antigen PA containing four of polypeptide as depicted in SEQ ID NO: 5 linked in parallel was synthesized, with a structure shown as follows:

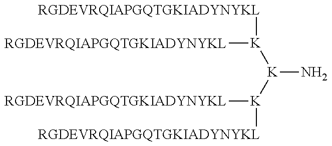

8.1.2 Based on an linking arm for parallel linking being one or more selected from the group consisting of Map-Lys(Map), Map-Orn(Map), Map-2,3-diaminopropionic acid(Map), Map-2,4-diaminobutyric acid(Map), Map-2,7-diaminoheptanoic acid(Map), Map-2,8-diaminooctanoic acid(Map), Map-Lys(Map)-Lys(Map-Lys(Map)), Map-Lys(Map)-Orn(Map-Lys(Map)), Map-Lys(Map)-2,3-diaminopropionic acid(Map-Lys(Map)), Map-Lys(Map)-2,4-diaminobutyric acid(Map-Lys(Map)), Map-Lys(Map)-2,7-diaminoheptanoic acid(Map-Lys(Map)) and Map-Lys(Map)-2,8-diaminooctanoic acid(Map-Lys(Map)), the polypeptides above were further introduced with Cys at their C-terminal, respectively, and preparations of these polypeptides were the same as synthesis of the linear polypeptide as described above, as well.

The resulting linear polypeptides were reacted directly with the amino acids of the linking arms at pH7, so as to obtain products after purification and frozen-drying.

A polypeptide antigen PB containing four of polypeptide as depicted in SEQ ID NO: 111 linked in parallel and a polypeptide antigen PB containing four of polypeptide as depicted in SEQ ID NO: 13 linked in parallel were synthesized, with a structure shown as follows:

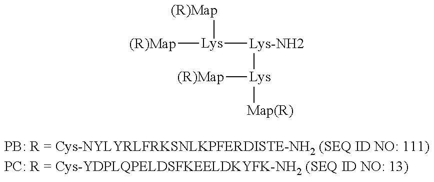

### 8.2 Animal Immunization

Animals, i.e. New Zealand white rabbits purchased from Qingdao Kangda Biotechnology Co., Ltd, were the first class animals passing quarantine without pathogenic bacteria, for each weighed about 1.5 kg. After one week's observation, rabbits were healthy and lively, with shiny fur and normal diet. These rabbits were then performed with immunity, and the specific immunity treatments were as follows.

Each branched polypeptide prepared in Example 2 was mixed with complete Freund's adjuvant (basic immunization) or incomplete Freund's adjuvant (booster immunization), fully emulsified, and injected intradermally into the back of rabbits at multiple points, with the total amount injected into per rabbit not exceeding 1.5 ml, containing about 1 mg of the branched polypeptide. Each branched polypeptide antigen was injected into three rabbits (as immunological replications) once every 3 weeks for 4 times, for each rabbit. On the 10th day after the fourth immunization, blood was collected from ear vein of the animals, for separating serum to perform determination. Additionally, a group treated with branched polypeptide alone and a group treated with adjuvant alone were set as control groups, where animals in the group treated with branched polypeptide alone were injected with branched polypeptide mixed with physiological saline which replaces the complete Freund's adjuvant, with respective amount equal to that in the branched polypeptide group, and the group treated with adjuvant alone was injected with complete Freund's adjuvant or incomplete Freund's adjuvant with its original concentration, with an equal amount to the final solution injected in the branched polypeptide group. And the groups treated with branched polypeptide alone and adjuvant alone were performed with the same administration manner and frequency as the branched polypeptide group.

### 8.3 Antibody Binding Assay

Antibody binding titer of each branched polypeptide was determined through an ELISA plate coated with such a branched polypeptide. Specific steps were as follows.

1) Preparing coating solution: 100 μl of a branched polypeptide solution (2 mg/ml) was mixed evenly with 100 ml of carbonate buffer solution at 0.05 M, to obtain a coating solution with concentration of 2 μg/ml. And blank control and negative control were set.

2) To each well of the ELISA plate, 100 μl of the prepared polypeptide solution were added, reacting at 4° C. overnight. After that, the liquid in the well was discarded.

3) Blocking in ELISA wells: with each well, a blocking reagent of 5% calf serum was fully filled and blocked at 37° C. for 40 min, after removal of bubbles in each well.

4) Washing: for each well, the liquid therein was removed and the wells were fully filled with a washing solution for washing for 3 min with slight shaking. Then, the liquid in the wells was poured off and the plate was patted dry on absorbent paper. This step was performed 3 times.

5) Adding samples: samples for test were introduced into a gradient dilution plate to establish appropriate concentration gradients, such as 1:500, 1:2000, 1:8000, 1:32000, 1:128000, 1:512000, and 1:2048000. The diluted samples were added into the wells of the ELISA plate, with 3 wells for each sample and 100 μl for each well, and incubated at 37° C. for 60 min. Then the wells were washed with the washing solution fully filled for 3 times, with 3 min each time.

6) Adding enzyme-linked antibody: goat anti-rabbit IgG labeled with horseradish peroxidase (ZSGB-BIO) as a secondary antibody, was diluted with PBS (pH 7.4) at a ratio of 1:20000, and 100 μl of the diluted secondary antibody were added into each well for incubating at 37° C. for 60 min. Then the wells were washed with the washing solution fully filled for 3 times, with 3 min each time.

7) Chromogenic reaction: taking tetramethyl benzidine (TMB) as a chromogenic reagent, 100 μl of TMB/H2O2-urea solution were added to each well and incubated at 37° C. for 3-5 minutes in the dark, and then added with 50 μl of stop buffer (sulfuric acid solution at 2 M) for each well to stop the reaction.

8) Detection: the optical density at 450 nm was determined within 20 min after stopping the chromogenic reaction.

The results are shown in Table 3. It can be seen that with immunization to the rabbits by the 3 branched polypeptide antigens individually, they all produced antibodies against respective branched polypeptides in serum, with high titers for antibodies more than 10³. No titer was detected in the group treated with branched polypeptide alone or adjuvant alone.

TABLE 3
Characteristics and immunizing titers of polypeptides repeated in the branched
polypeptide antigen (with Freund's adjuvant)

| | Positions of | | Titers of |
|---|---|---|---|
| Branched | the epitope | Sequence of | polypeptide |
| polypeptide | polypeptide | repeated epitope polypeptide | antigen binding |
| PA | S403-425 | RGDEVRQIAPGQTGKIADYNYKL | 8000 |
| PB | S450-471 | NYLYRLFRKSNLKPFERDISTE | 4000 |
| PC | S1138-1157 | YDPLQPELDSFKEELDKYFK | 2000 |

Although embodiments have been described above, it should be understood that the embodiment of the present disclosure is not limited thereby. Instead, the embodiments of the present disclosure comprise all the modifications, alternatives, and improvements within the spirit and scope of the present disclosure.

1-10. (canceled)

11. A polypeptide vaccine, comprising:
one or both of polypeptide antigens as depicted in SEQ ID NOs: 13 and 111; and
an adjuvant compatible with the polypeptide antigen.

12. The polypeptide vaccine of claim 11, wherein the polypeptide vaccine comprises the polypeptide antigen as depicted in SEQ ID NO: 13.

13. The polypeptide vaccine of claim 11, wherein the polypeptide vaccine comprises the polypeptide antigen as depicted in SEQ ID NO: 111.

14. The polypeptide vaccine of claim 11, wherein a plurality of the polypeptide antigens are presented in series or in parallel, forming a recombinant protein.

15. The polypeptide vaccine of claim 14, wherein the plurality of the polypeptide antigens in the formed recombinant protein are linked via a linking arm therebetween.

16. The polypeptide vaccine of claim 15, wherein the linking arm for series linking is one or more selected from the group consisting of Gly, Lys, AEA, Ava, ANP, beta-Ala, GAB and PEG.

17. The polypeptide vaccine of claim 15, wherein the linking arm for parallel linking is one or more selected from the group consisting of Lys, Orn, 2,3-diaminopropionic acid, 2,4-diaminobutyric acid, 2,7-diaminoheptanoic acid, 2,8-diaminooctanoic acid, Map-Lys(Map), Map-Orn(Map), Map-2,3-diaminopropionic acid(Map), Map-2,4-diaminobutyric acid(Map), Map-2,7-diaminoheptanoic acid(Map), Map-2,8-diaminooctanoic acid(Map), Map-Lys(Map)-Lys(Map-Lys(Map)), Map-Lys(Map)-Orn(Map-Lys(Map)), Map-Lys(Map)-2,3-diaminopropionic acid(Map-Lys(Map)), Map-Lys(Map)-2,4-diaminobutyric acid(Map-Lys(Map)), Map-Lys(Map)-2,7-diaminoheptanoic acid(Map-Lys(Map)) and Map-Lys(Map)-2,8-diaminooctanoic acid(Map-Lys(Map)).

18. The polypeptide vaccine of claim 14, wherein the formed recombinant protein comprises the polypeptide antigen repeated in series or in parallel for once to 10 times.

19. The polypeptide vaccine of claim 14, wherein the formed recombinant protein comprises the polypeptide antigen repeated in series or in parallel for 4 times.

20. The polypeptide vaccine of claim 14, wherein the formed recombinant protein comprises the plurality of the polypeptide antigens and 4 to 6 of His or 4 of Gly and 1 of Ser.

21. The polypeptide vaccine of claim 14, wherein the plurality of polypeptide antigens in the formed recombinant protein are linked via a carrier protein therebetween.

22. The polypeptide vaccine of claim 21, wherein the carrier protein is selected from a group consisting of bovine serum albumin, ovalbumin, keyhole limpet hemocyanin and casein.

23. The polypeptide vaccine of claim 21, wherein the carrier protein is keyhole limpet hemocyanin.

24. The polypeptide vaccine of claim 11, wherein the polypeptide antigen is conjugated with a carrier protein.

25. The polypeptide vaccine of claim 24, wherein 5 to 50 of polypeptide antigens are conjugated with each carrier protein.

26. The polypeptide vaccine of claim 11, wherein the adjuvant is selected from a group consisting of aluminum hydroxide, Corynebacteriuum parvm, lipopolysaccharide, cytokines and alum.

27. The polypeptide vaccine of claim 11, wherein the adjuvant is a complete Freund's adjuvant or incomplete Freund's adjuvant.

28. A method for preventing or treating a coronavirus infection or a disease caused by the coronavirus, with a polypeptide vaccine of claim 11.

29. The method of claim 28, wherein the coronavirus is selected from a group consisting of SARS-CoV, MERS-CoV and SARS-CoV-2.

30. The method of claim 28, wherein the coronavirus is SARS-CoV2.
